(19) United States
(12) Patent Application Publication
(10) Pub. No.: US 2019/0057182 A1
(43) Pub. Date: Feb. 21, 2019

(54) BIOMARKER-DRIVEN MOLECULARLY TARGETED COMBINATION THERAPIES BASED ON KNOWLEDGE REPRESENTATION PATHWAY ANALYSIS

(71) Applicant: CSTS HEALTH CARE INC., Toronto (CA)

(72) Inventors: Giannoula Lakka Klement, Boston, MA (US); Ali Hashemi, Toronto (CA); Thomas Getgood, Toronto (CA); Christos Klement, Toronto (CA); Edward A. Rietman, Nashua, NH (US)

(73) Assignee: CSTS HEALTH CARE INC., Toronto, ON (CA)

(21) Appl. No.: 15/576,543

(22) PCT Filed: May 24, 2016
(86) PCT No.: PCT/CA2016/050586

§ 371 Date: Nov. 22, 2017

Related U.S. Application Data
(60) Provisional application No. 62/165,879, filed on May 22, 2015.
(60) Provisional application No. 62/194,090, filed on Jul. 17, 2015.

(30) Foreign Application Priority Data
May 20, 2016 (CA) PCT/CA2016/050581

(51) Int. Cl. G06F 19/12; G16H 50/20; C12Q 1/6886; G06N 5/02; G06F 19/18; G06F 19/16

(52) U.S. Cl. CPC G06F 19/12; G16H 50/20; C12Q 1/6886; C12Q 2600/106; G06F 19/18; G06F 19/16; C12Q 2537/165; G06N 5/02

(57) ABSTRACT

A method for therapeutic application involves accessing information associated with a patient and a reference biological network database, generating, using the information associated with the patient and the reference biological network database, a disease model, identifying, from the disease model, a molecular target, identifying, from the molecular target, a drug for the patient, generating, based on the drug for the patient, a treatment plan for the patient, and repetitively generating, based on repetitively inputting a patient outcome from the treatment plan into a feedback loop mechanism, a different treatment plan for the patient based on either the molecular target or a different molecular target.

### BACKGROUND

In recent years, the falling cost and increased availability of genetic testing has allowed oncology treatments to be increasingly informed by specific molecular alterations of the patients and their cancer., However, establishing the oncological relevance of a given molecular alteration (mutation, variation, over-expression, down-regulation or other) is notoriously difficult. In addition, the dominant diagnostic paradigm to date has been based on histology and the site of occurrence (i.e. breast, lung etc.). Moreover, if a molecular finding is made on the basis of the pathologist's suspicion, then only known molecular targets are considered.

At a high-level, the treatment decision is presently made for most patients on the basis of a histopathology—that is to say, the standard of care or regimen provided to the patient will be driven by the disease site-specific diagnosis. This means that even though there are genetically different subtypes of breast cancer, all of these will be grouped together by virtue of shared body site.

Based on the premise that more chemotherapy kills more cancer cells, most standard of care treatments follow the “Maximum Tolerated Dose” (MTD) approach as described by Skipper et al. in a 1970 publication titled “Implications of biological cytokinetic, pharmacologic, and toxicologic relationships in design of optimal therapeutic schedules,” published in volume 54 of Cancer Chemotherapy Reports, Skipper et al. formulated the basic rational for MTD as the maximum amount of drug or radiation that we can give patient without killing them.

In contrast, in recent years, there is an increasing push towards metronomic therapies, low-dose frequent chemotherapy, particularly when combined with biological agent as taught by Andre et al. in a 2014 publication titled “Metronomics: towards personalized chemotherapy?” in volume 11 issue 7 of Nature Reviews Clinical Oncology and by Kareva et al in a 2015 publication titled “Metronomic chemotherapy: an attractive alternative to maximum tolerated dose therapy that can activate anti-tumor immunity and minimize therapeutic resistance,” in volume 358 issue 2 of Cancer Letter.

Unlike the traditional maximum tolerated chemotherapy (MTD), low-dose frequently administered chemotherapy (metronomic) preserves the eco-evolutionary forces within the tumor microenvironment as summarized recently by Klement in a 2016 publication titled “Eco-evolution of cancer resistance” in volume 8 issue 327 of Science Translational Medicine.

Metronomic chemotherapy should therefore represent a surrogate for any form of low-dose chemotherapy administration that targets tumor microenvironment (as opposed to the cancer cell itself). It should include “adaptive therapy” described by Robert Gatenby in a 2009 publication titled “Adaptive Therapy” in volume 69 of Cancer Research, “dose-dense therapy as described by Fornier et al in 2005 publication titled “Dose-dense adjuvant chemotherapy for primary breast cancer” in volume 7 issue 2 of Breast Cancer Research and other forms of low-dose chemotherapy which are optimal for combination with targeted agents.

Increasing evidence exists that the traditional histology based diagnosis is inadequate, and much is to be gained by considering the molecular signature of the disease. Namely, Hoadley et al. describes in a 2014 publication titled “Multiplatform Analysis of 12 Cancer Types Reveals Molecular Classification within and across Tissues of Origin,” in Volume 158 of Cell that one can design cheaper, more effective therapies by considering the specific, often unique molecular alterations that have occurred in each patient and their cancers. Based on these recent findings, many oncologists look to incorporate genetic information into their clinical decision making.

As noted above, the use of this information spans the gamut from:
1. a populational guess (e.g. given that I know that the patient has Breast Cancer, and 80% of Breast Cancers are driven by a mutation in BRCA, I will therefore target BRCA);
2. to testing for specific mutations—e.g. given that the patient has breast cancer, and HER2 is a known driver imitation, I will order a test to see if this mutation is present;
3. to testing for a panel of mutations—e.g. test for ˜600 genes known to be associated with cancer progression to see which of these mutations are present in the patient; and
4. to testing the entire genome/transcriptome—e.g. to see which genes are altered or strongly up/down regulated in both the patient and the cancer.

Many researchers and oncologists are striving to differentiate between drivers and passengers when looking at expression or mutational analysis of various cancers. Most presently employed candidate gene panels look for alterations only in genes that have been suspected in the literature and other authoritative sources to be driver genes. This approach has an inherent bias for genes and proteins that have been “around” for a long time (early discoveries such as p53, HER2 or EGFR), rather than for those targets that most affect pathways involved in disease progression. Many of the later may not have been identified yet. While only using literature validated targets may help alleviate the information glut, the approaches are based on insufficient information given our relative paucity and incomplete knowledge of the role that genetic alterations may play in the host and cancer biology, and such an approach is likely to lead to suboptimal therapies.

The complexity and difficulty of applying genomic testing in a clinical setting increases as the sophistication of tests increase. Indeed, at this point in time, a key difficulty in the field is how to interpret the results of genomic testing. To this end, a number of players have come out, providing partial solutions. For example, the company Foundation Medicine in a 2014 publication titled “System and Method for Managing Genomic Testing Results,” offers a candidate-based approach to genomic testing, and has developed a “molecular information product”, that helps match genetic alterations with ongoing clinical trials. In this way, Foundation Medicine helps clinicians select a clinical trial which will target a single molecule in the patient. Another company, Molecular Health, has developed a bioinformatics platform to aid their Medical Director in again, selecting the appropriate single target.

In other cases, the Van Andel Research Institute has developed a patent, U.S. Pat. No. 7,660,709, to select a single molecular target based on a hypergeometric statistical analysis of the protein-protein interaction (PPI) networks of the mutations. Lastly, IBM in their adaptation of their Watson technologies to oncology, crunches much of the available literature, textbooks and other sources to recommend a single molecular target for the oncologist.

From one point of view, while the solutions mentioned above are a step in the right direction, they are all limited to single target therapies.

### SUMMARY

A computer-implemented method for therapeutic application including the steps of accessing information associated with a patient and a reference biological network database, generating, using the information associated with the patient and the reference biological network database, a disease model, identifying, from the disease model, a molecular target, identifying, from the molecular target, a drug for the patient, generating, based on the drug for the patient, a treatment plan for the patient, and repetitively generating, based on repetitively inputting a patient outcome from the treatment plan into a feedback loop mechanism, a different treatment plan for the patient based on either the molecular target or a different molecular target.

A computing system for therapeutic application, including a processing module comprising a computer processor with circuitry configured to execute instructions configured to: access information associated with a patient and a reference biological network database, generate, using the information associated with the patient and the reference biological network database, a disease model, identify, from the disease model, a molecular target, identify, from the molecular target, a drug for the patient, generate, based on the drug for the patient, a treatment plan for the patient, and repetitively generate, based on repetitively inputting a patient outcome from the treatment plan into a feedback loop mechanism, a different treatment plan for the patient based on either the molecular target or a different molecular target.

A non-transitory computer-readable medium having instructions stored thereon that, in response to execution by the computer system, cause the computer system to perform operations including: accessing information associated with a patient and a reference biological network database, generating, using the information associated with the patient and the reference biological network database, a disease model, identifying, from the disease model, a molecular target, identifying, from the molecular target, a drug for the patient, and generating, based on the drug for the patient, a treatment plan for the patient, repetitively generating, based on repetitively inputting a patient outcome from the treatment plan into a feedback loop mechanism, a different treatment plan for the patient based on either the molecular target or a different molecular target. Other aspects and advantages of the invention will be apparent from the following description and the appended claims.

### BRIEF DESCRIPTION OF DRAWINGS

FIG. 1 shows a diagram in accordance with one or more embodiments.

FIGS. 2A and 2B show a flow chart in accordance with one or more embodiments.

FIG. 3 shows a diagram in accordance with one or more embodiments.

FIG. 4 shows a flow chart in accordance with one or more embodiments.

FIGS. 5A and 5B show a computing system in accordance with one or more embodiments.

FIG. 6 shows a schematic diagram in accordance with one or more embodiments.

### DETAILED DESCRIPTION

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

Throughout the application, ordinal numbers (e.g., first, second, third, etc.) may be used as an adjective for an element (i.e., any noun in the application). The use of ordinal numbers does not imply or create a particular ordering of the elements nor limit any element to being only a single element unless expressly disclosed, such as by the use of the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a horizontal beam” includes reference to one or more of such beams.

Terms like “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Although multiple dependent claims are not introduced, it would be apparent to one of ordinary skill in that that the subject matter of the dependent claims of one or more embodiments may be combined with other dependent claims. For example, even though claim 3 does not directly depend from claim 2, even if claim 2 were incorporated into independent claim 1, claim 3 is still able to be combined with independent claim I that would now recite the subject matter of dependent claim 2.

In one or more embodiments, this invention describes a computationally-driven oncology therapy design strategy that draws upon multiple fields of science. The computationally-driven oncology therapy design strategy is based on a molecular analysis of the patient and of the patient tumor and is able to provide recommendations for a metronomic, bio-marker driven, molecularly targeted combination therapy.

In the medical research area, the term “molecular” includes genomic and proteomic assays that uses whole genome sequencing (WGS), messenger RNA (mRNA), and clustered regularly interspaced short palindromic repeats (CRISPR).

In one or more embodiments, one or multiple molecular targets may be employed. Embodiments are built around a central feedback loop mechanism for utilizing patient and population outcome data, complemented by a continual monitoring of new published and reliable information to inform future therapy decisions.

In one or more embodiments, interpretation of the gene/protein expression analysis (transcriptome, proteome, exome, metabolome, or other form of molecular information) of a tumor sample taken from a patient is relied on, via an understanding of different branches of science as realized in a knowledge representation software system and constantly updated by a number of machine learning algorithms and informed by all previous decisions made by clinicians and other specialists using the system, patient outcomes, and new insights from literature monitored by the system.

Specifically, in general, embodiments of the invention are directed toward a system and method that allows a health care provider, assisted by computer technologies and technical acquisition techniques to integrate relevant available information and interactively build a patient-specific model of the disease. This patient-specific model of cancer or other molecularly driven disease is then used to instantiate a unique therapy based on the oncology therapy design strategy embedded in the system and allow previous clinical decisions and learning to optimize a given patients treatment strategy.

Different types of targeted therapeutic strategies can be summarized into the following categories:
1. Targeted therapies that target a specific, single molecule solely based on previously published data about the presence of a molecular alteration having a role in the cancer on the basis of population statistics (candidate molecule target clinical trial for a specific tissue type);
2. Targeted therapies that test for a specific molecule, given the histology of the tumor, and target a specific, single molecule if the mutation is present (candidate clinical trial inclusive only of patients positive for the target);
3. Targeted therapies that test for a panel of candidate molecules (usually established oncogenes), yet treat a single, specific target, either based on the availability of a clinical trial or on the approval of a regulatory agency such as the Food and Drug Administration (FDA) or European Medicines Agency (EMA) (considered personalized or individualized);
4. Targeted therapies that test the entire transcriptome of the tumor and/or patient, and select a single molecular target (considered personalized or individualized); and
5. Targeted therapies that test molecular information such as transcriptome, proteome, exome and/or other molecular information (the candidate approach is a subset of the full transcriptome) and select a combination of molecular targets according to the ‘pathway activation strategy’.

In one or more embodiments, gene/protein expression analysis from the patient's tumor and on any additional information about the tumor biology (phosphorylation, methylation arrays etc.) is used. In one or more embodiments, a metronomic, biomarker-driven, molecularly targeted combination therapy uniquely for each patient is generated.

The system of one or more embodiments works with as much molecular information as available (a full transcriptome of the tumor; substractive transcriptome of tumor tissue and patient normal tissue; proteomic analysis of the same; metabolomics information such as phosphorylation or methylation; pharmacogenomics information etc.), though at a minimum, the system of one or more embodiments requires genomic information in the form of gene expression (transcription) microarrays or a large panel of genomic alterations.

It would be apparent to one of ordinary skill in the art that generally the more genes present in the panel, the better. To exploit the full potential of the system of one or more embodiments, ideally a complete transcriptome would be used for analysis. However, the system of one or more embodiments functions properly without a complete transcriptome.

It would be further apparent to one of ordinary skill in the art that while the advent of genomic testing—whether by a panel of genes or the entire genome—offers tremendous potential in clinical decision-making, a dearth of options exist regarding how to interpret and apply this information for clinical application. The end-to-end process, starting from diagnosis to the design and administration of therapies, should be considered to fully understand the system and method of one or more embodiments.

In accordance with one or more embodiments, an emphasis is placed on the use of wide ranging molecular information (transcriptome, proteome, genome, metabolome etc.) of the patient (with candidate genomic testing as a subset of the approach) and situating all of these approaches in the continuously curated and academically validated PPI networks.

In one or more embodiments, the specific strategy of integrating all available information from disparate disciplines and sources in a single system, which works in conjunction with clinicians to design a metronomic, bio-marker driven, molecularly targeted combination therapy is utilized.

It would be apparent to one of ordinary skill in the art that although cancer is used as an illustrative disease throughout the rest of this document, one or more embodiments are applicable for any disease where molecular information can he obtained. Accordingly, one or more embodiments should not be limited to any single disease or example.

It would be further apparent to one of ordinary skill in the art that the therapy described as part of one or more embodiments is metronomic in that the targets of the biologically optimized low-dose frequent chemotherapy is the tumor microenvironment.

Furthermore, the therapy described as part of one or more embodiments is bio-marker driven, in that the clinical decisions are based on the presence of molecular alterations found in the patient's cancer via molecular testing. Furthermore, the therapy described as part of one or more embodiments is molecularly targeted in that the therapy selects drugs which modulate a specific molecular target identified by this novel strategy as being key to disrupting cancer progression.

Even further, the therapy described as part of one or more embodiments is a combination therapy in that the therapy realizes that targeting a single molecule is often inadequate due to the many alternate pathways or reaction chains protecting survival and growth, pathways in cells. Consequently, the system of one or more embodiments will propose therapies with more than one molecular target.

It would be apparent to one of ordinary skill in the art that an underlying assumption in the approach described as part of one or more embodiments is that single alterations rarely account for the complexity of cancer biology, and many developmental pathways are re-activated rather than mutated in cancer.

This assumption is shown in in TABLE 1 below. TABLE 1 includes selected examples of signaling pathways illustrating the necessity of a bio-marker-driven, pathway analysis informed, therapy design. As seen in TABLE 1, a single drug approach assumes a single alteration and absence of alternative pathways (Left). This scenario is rarely the case and multiple agents may be needed for full inhibition when there is more than a single alteration (Middle), and when the pathways merge at at least one point. However, multiple targets should be submitted to pathway analysis, as many converge on a single target and others diverge into alternative pathway(s) (Right). Combinatory therapies appear to be, therefore, the most rational approach.

Therefore, targeting a single alteration is unlikely to be effective in combatting the disease. In contrast, one or more embodiments are predicated on the fact that cancers generally repurpose normal biological pathways via a set of molecular alterations at gene or protein level. Accordingly, the biological effect induced by the molecular alteration, and which biological pathway(s) have been “hijacked” by the cancer should be considered and determined.

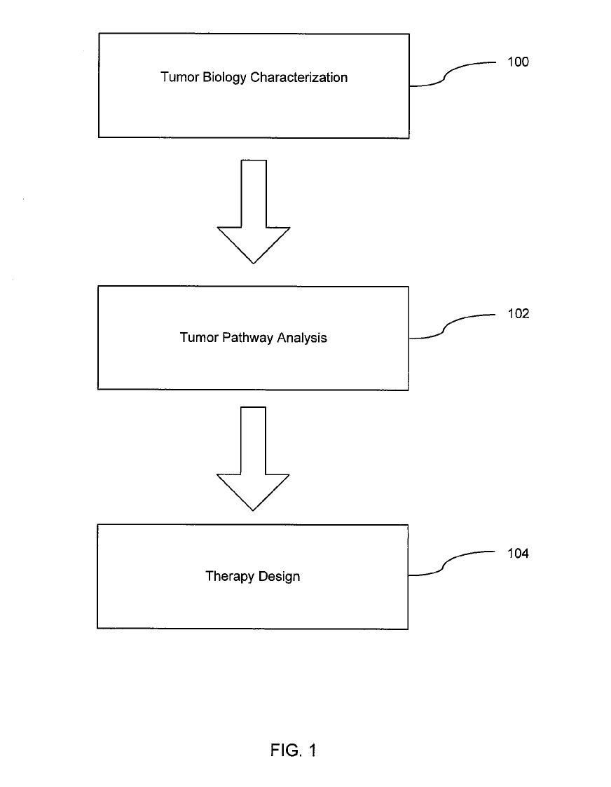

In view of the above, FIG. 1 shows a diagram in accordance with one or more embodiments. As seen in FIG. 1, the strategy of the targeted therapy used in one or more embodiments is divided into three main domains: Tumor Biology Characterization (100), Tumor Pathway Analysis (102), and Therapy Design (104).

In one or more embodiments, the Tumor Biology Characterization domain (100) is based on the latest understanding of cancer as an ecosystem with multiple populations of heterogeneous subpopulations of cells with varied levels of drug resistance, angiogenesis potential, immune evasiveness and invasiveness. The Tumor Biology Characterization (100) considers host (microenvironment) changes as well as the dominant tumor cell population.

In one or more embodiments, the Tumor Pathway Analysis domain (102) is characterized by the known protein-protein interactions, but this domain is constantly updated as new information emerges from peer-reviewed scientific literature. Instead of the presently used bioinformatics approach, which is focused on identifying the frequency of specific genomic changes in a patient population, the system described herein provides the first meaningful overlay of specific patient information on the interaction networks (PPIs).

Similarly, in one or more embodiments, the Therapy Design domain (104), which is also constantly updated through scientific literature, is being employed here in a unique setting. Instead of remaining within the domain of the pharmacologists and pharmacists looking for host toxicities and pharmacodynamics, the system is computer empowered to consolidate upstream and downstream information from pathways. The system is further empowered and informed by each individual response within a patient and within a population to find drugs that may not be direct inhibitors of a genomic alteration but may act downstream from it. The combination of the three domains represents an invention capable of analyzing and utilizing information from individual treatments, incorporating those treatments into a therapeutic design, informing future therapies with past failures/successes, and providing clinical guidelines based on multiple N=1 trials accumulated over time.

In one or more embodiments, the Tumor Biology Characterization (100) domain includes the conduction of a full, both transcriptome and sequencing, cancer genome and host genome analysis. Furthermore, in one or more embodiments, the Tumor Biology Characterization domain also includes an immunohistochemistry identification of known proteins of interest and an identification of epigenetic and environmental factors.

In one or more embodiments, the Tumor Pathway Analysis (102) domain shown in FIG. 1 includes the understanding of gene alteration(s) and the altered gene's new biological function and the effect of the alteration on the PPI network(s). Furthermore, in one or more embodiments, the Tumor Pathway Analysis (102) domain also includes the understanding of how these changes in the tumor pathways impact the host.

In one or more embodiments, the Therapy Design (104) domain includes the factors that go into designing the treatment plan for the patient. These factors include: selecting the minimum number of gene alteration(s) needed to inhibit pathways associated with cancer progression, monitoring the patient's outcomes and adjusting the therapy accordingly, capturing outcome information from patients treated with targeted therapies in order to inform future therapeutic decisions, providing large body of evidence to inform future therapeutic decisions, establishing pharmacodynamics and pharmacogenetics of combinations of targeted agents, and providing affordable and accessible therapies to the patient.

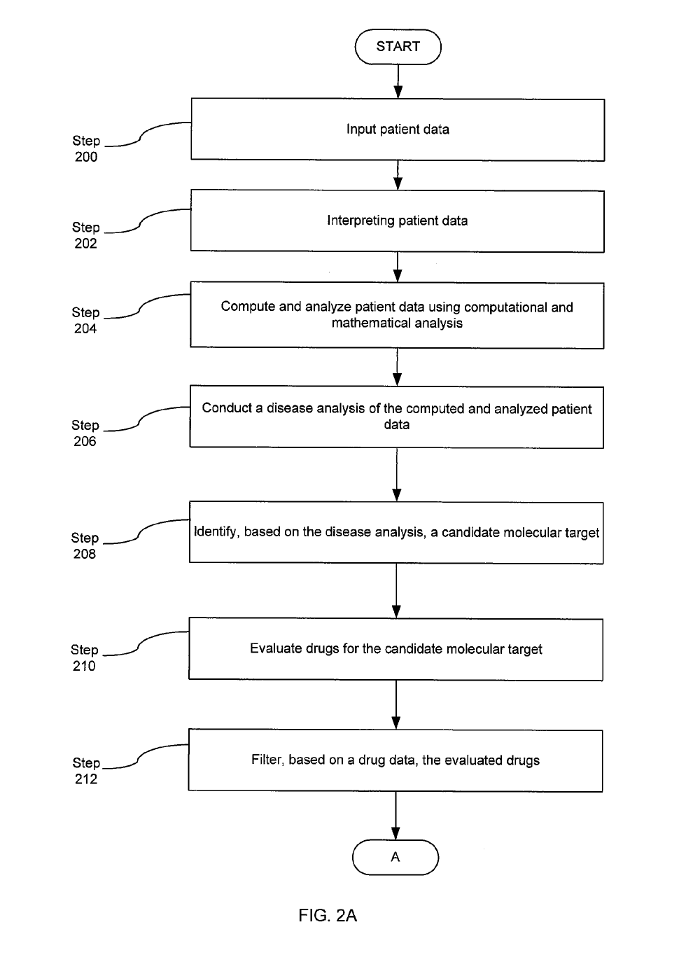

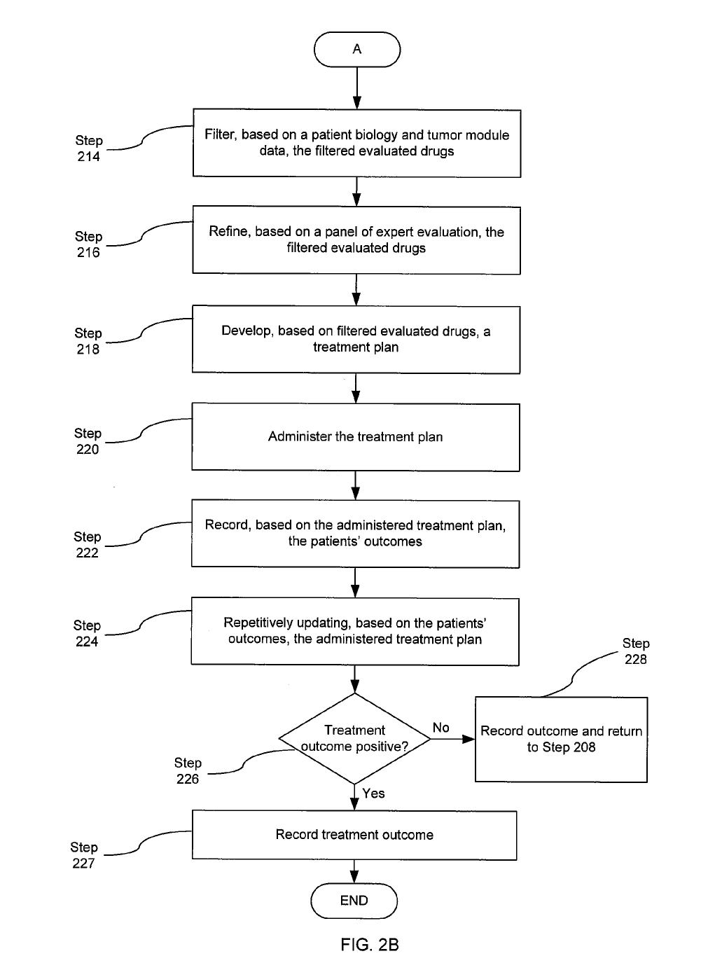

FIGS. 2A and 2B show a flow chart of a method in accordance to one or more embodiments. In one or more embodiments, the method in the flow charts in FIGS. 2A and 2B involve inputting patient data (Step 200), interpreting patient data (Step 202), computing and analyzing patient data using computational and mathematical analysis (Step 204), conducting a disease analysis (also referred to as a disease model) of the computer and analyzed patent data (Step 206), identifying, based on the disease analysis, a candidate molecular target (Step 208), evaluating drugs for the candidate molecular target (210), filtering, based on a drug data, the evaluated drugs (Step 212), filtering, based on a patient biology and tumor module data, the filtered evaluated drugs (Step 214), refining, based on a panel of expert evaluation, the filtered evaluated drugs (Step 216), developing, based on the filtered evaluated drugs, a treatment plan (Step 218), administering the treatment plan (Step 220), recording, based on the administered treatment plan, the patient outcomes (Step 222), repetitively updating, based on the patient outcomes, the administered treatment plan (Step 224), determining if the treatment outcome is positive or negative (Step 226), and recording the positive and negative outcome for informing therapy for future patients having involvement of the same pathway(s) and requiring therapy (Step 227).

In one or more embodiments, dependent of the results of Step 226, if the outcome of the treatment plan is not positive, the failure data is recorded and the particular agent is ranked lower (less evidence for its efficacy) in future therapeutic recommendation for patients with similar molecular signatures and the method returns to Step 208 (Step 228) in order to refine future therapy designs based on the results of the current therapy or therapies. In one or more embodiments, if the outcome of the treatment plan is positive, the successful data is recorded and the particular agent is ranked higher ore evidence for it efficacy) (Step 227).

The steps in the flow chart of FIGS. 2A and 2B for the method of suggesting combination therapies described as part of one or more embodiments is further described as follows:
1. The system of one or more embodiments takes as input molecular information—ideally including rare transcripts, splice variants, fusion transcripts, gene expression analysis, protein analysis, metabolic information (such as phosphorylation or methylation) and other modes of finding molecular alterations in both the tumor tissue and the patient. At minimum, the system of one or more embodiments needs to take as input a set of genes and their expression levels.
2. The system of one or more embodiments then maps this information into its disease interpretation knowledge base to anchor into protein-protein interaction and biological pathway networks culled from multiple data sources.
3. The system of one or more embodiments weighs the available networks according to the unique composition of the patient's unique molecular signature. Specifically:
a. The system of one or more embodiments gives preference to subnetworks where multiple altered genes/proteins have been identified or are highly active.
b. The system of one or more embodiments gives additional weight to genes that are known oncogenes as established by peer-reviewed literature or other reliable sources.
c. The system of one or more embodiments gives additional weight to pathways which are known and associated with various cancers as established by peer-reviewed literature or other reliable sources.
4. The system of one or more embodiments then presents a series of PPI-networks corresponding to biological pathways which may be induced by the combination of genetic mutations and variants to be supporting the disease.
5. The system of one or more embodiments then analyzes the structure of these resultant networks to identify molecular targets which may in combination be best suited to combat the disease.
a. Specifically, the system of one or more embodiments applies a series of thermodynamic and mathematical analyses to further rank the importance of specific proteins within the protein-protein interaction networks given the expression levels, as well as topological and flow analyses.
b. These analyses are connected to the system of one or more embodiments in a plug-in manner, with each different thermodynamic or mathematical approaches yielding different scorings for the gene-protein networks.
c. Additionally, a meta-reasoner aggregates results from the different plug-in analyses to yield the best potential set of therapeutic targets.
6. The system of one or more embodiments then scours available literature, or other reliable sources, such as private, public, and academic databases, to find known drugs which can target the identified pathway(s) or molecular target(s). It has the following preference criteria for how to present drug information:
a. The system of one or more embodiments utilizes a minimal set of drugs to target the set of host and molecular alterations to combat the disease. If more than one equivalent drug is found, these are listed in order from lowest to highest in cost and availability.
b. The system of one or more embodiments strongly prefers agents which are Food and Drug Administration (FDA), European Medicines Agency (EMA) (or equivalent regulatory body, depending on the jurisdiction(s) involved) approved for the disease indication.
c. The system of one or more embodiments prefers agents which are FDA, EMA (or equivalent regulatory body) approved but for other disease indications.
d. The system of one or more embodiments also considers agents which are still experimental but affect the selected molecular targets.
7. The system of one or more embodiments simultaneously considers any of the host's secondary conditions from the available medical record to filter out harmful or ineffective therapies.
8. The system of one or more embodiments simultaneously considers any gene variants which are known to render certain drugs ineffective (pharmacogenomics).
9. The system of one or more embodiments simultaneously considers known combination therapies and notes any evidence that support/counter a given drug/gene combination.
10. The system of one or more embodiments then cross-references the resultant genetic databases and networks, selected targets and possible therapies, and filters out combinations with known phenotypes which may render a potential treatment ineffective or dangerous.
11. The system of one or more embodiments additionally considers the available evidence (literature, databases and other reliable sources) for potentially toxic drug-drug interactions or known dosage frequency limits for the drugs, and additionally filters out the set of drugs.
12. The system of one or more embodiments additionally considers the cost of the drugs and the anticipated health care costs associated with the use of the drug or drug combination (hospitalization vs. in hospital care), and additionally filters out the set of agents to derive and optimize for the most effective therapy at minimal cost.
13. The system of one or more embodiments collects and stores information about the decisions caregivers made, and correlates these choices with the related outcomes in order to inform future therapeutic decisions with this stored evidence of efficacy.
14. The system of one or more embodiments then presents to the clinician:
a. A set of gene-gene and protein-protein interaction subnetworks which are likely to be supporting disease progression;
b. Highlights the altered molecular changes within these networks;
c. Identifies potential therapeutic agents that can be used in combination; and/or
d. A set of rationale, outlining step-by-step the decision making process, and ultimately linking back to collected body evidence which support the present operational model of the disease. Namely presenting:
   1. Literature and other evidence to support the selected networks relevant to therapy;
   2. Evidence, including mathematical analyses to support the selected molecular targets; and/or
   3. Evidence and the chain of reasoning behind the drug selections.
15. The system of one or more embodiments using its Disease Interpretation Knowledge Model then automatically generates English (or other) natural language descriptions and documents highlighting the rationale behind the suggested target molecular networks.
16. In this way, the system of one or more embodiments has a constructed an operational model of the cancer including potential cancer therapies that are unique to the patient's biology and molecular analyses.
17. This system of one or more embodiments can then be used by an expert panel, such as a tumor board, to validate and build upon the disease model and upon treatment recommendations.
18. Specifically, oncologists and other experts can use the system of one or more embodiments to evaluate the rationale behind each of the choices, and can introduce novel evidence or arguments to refine and extend the rationale and model of the disease.
19. Once the expert panel agrees on a reliable therapy, the clinician configures the treatment strategy for a given patient within the system of one or more embodiments.
20. As the treatment is administered to the patient, the patient's response is measured and the outcomes fed back into the system of one or more embodiments.
21. As patients respond to therapies, the system of one or more embodiments uses this additional novel input to (re)asses its rationale, building support for or against particular therapies.
22. One or more embodiments of the invention incorporates a feedback loop, whereby as new patient outcomes are collected, a set of proprietary algorithms analyze the new data, creating similarity profiles and continuously grouping sets of patients, genetic mutations and drugs into similarity groups.
a. In this way, the system of one or more embodiments updates its models of the patient and tumor biology, pathways and drug response.
b. Additionally, the system uses its similarity measures to monitor new patients, and provides feedback based on the collected patient outcomes to clinicians who are designing new therapies.
23. If patients are not responding to a therapy, the caregiver and/or the expert panel may revisit the active therapies and REFINE or FAIL a therapy based on patient outcome data or new literature/evidence.
a. In this instance, the therapy design process would begin again, taking into account novel evidence showing the ineffectiveness of a component of or the entire previous therapy design.

The system of one or more embodiments described above, and any method of using the system of one or more embodiments, makes use of key technologies in enabling its existence. The amount of information it covers is beyond the scope of any single individual, and represents the aggregate knowledge and input of experts in medicine, oncology, bioinformatics molecular biology, physics, mathematics and other disciplines.

It would be apparent to one of ordinary skill in the art that it is critical to stress the importance of the feedback loop mechanism of one or more embodiments described above as this mechanism allows information about the host, the known phenotypes, the molecular information & drug toxicities to inform therapeutic decisions about the individual patient.

It would further be apparent to one of ordinary skill in the art that iterative feedback is central to the combination therapeutic strategy. In one or more embodiments of this invention, the iterative feedback loops of one or more embodiments also provide both individual and populational statistics that allow prioritization of drug choices based on previous success or failure of combinatorial therapies. In addition, the inclusion of drug cost and health care cost in the decision making process, the system allows the caregiver to exercise fiscal responsibility without jeopardizing patient care.

In one or more embodiments, the system and methodology described herein is a complex socio-technical system that has been created to find the right combination and balance of a solution that combines software with people. In one or more embodiments of this invention, the diagram below shows why the system implementation of the strategy is considered a complex socio-technical system. The diagram shows how integrating knowledge from different specialties and allowing for cross-collaboration through knowledge representation tools, the opinion of a single physician is enhanced. Furthermore, the system of one or more embodiments is also built for continued evaluation of emerging data by mathematicians, physicists, knowledge engineers, programmers, and other bio-informatics and technologists.

As shown in TABLE 2 above, one or more embodiments uses techniques from the field of artificial intelligence to represent expert knowledge from numerous scientific disciplines to create a computational model of the disease, and uses this computational model of disease to align, organize and interpret the available information. Specifically, using knowledge representation techniques, an operational model of disease can be built.

It would be apparent to one of ordinary skill in the art that in a domain as complex as cancer, there is no individual who has a complete picture or model of how the cancer works, especially when viewed from the perspective of an individual specializing in a specific field of science (such as genomics, proteomics, metabolomics, pharmacogenomics, etc.) That is to say, while each expert has a partial model of how their area of expertise applies to our understanding of cancer, the aggregate, holistic view of how everything ties together is not available to a single person.

In one or more embodiments, an operational model of the disease (such as cancer) has been constructed that aggregates the expert knowledge—the partial models each expert holds—into a unified whole. In this way, one can say that the computer “understands” the disease at a level of completeness that is beyond a single person or a single scientific domain expert.

Using this model, online sources have been additionally identified such as databases, literature, and other content which contain relevant, reliable information. The operational disease model is thus used to interpret and align such available information, according to its relevance to this holistic understanding of disease. This model is used to map and bring together information from disparate sources in a view that is geared towards a clinicians understanding the relevance genomics, proteomics, metabolomics, etc. are relevant to making clinical decisions to design molecularly targeted therapies.

One or more embodiments of the method involve a caregiver or an expert panel, such as a tumor board, trying to design a personalized therapy for a patient. As assumption has been made that the patient has been diagnosed with a disease, and a test which includes at the very least transcription information for the patient's genes is available.

In one or more embodiments, the results of such tests are either manually entered into the system described above, or it is automatically read from test reports and results files, in digital form, and integrated into the patient file. In one or more embodiments, this instantiates an initial model for the patient's cancer. Specifically, the system of one or more embodiments uses this information to personalize the patient's genetic (and proteomic etc.) data into a set of protein-protein interaction (PPI) networks and biological pathway resources.

In one or more embodiments, the system begins by pin-pointing genes/proteins of interest, looking for high or low expression, displaying fusion genes and/or known genetic alterations, the system cross-references this information with its archived and real-time monitoring of literature and other authoritative sources.

In this manner, the system of one or more embodiments is able to filter and focus its attention on the set of molecular alterations most likely contributing to disease progression. Central to this process, the system of one or more embodiments analyzes the PPI graphs and protein neighbors on such graphs, applying a variety of topological and thermodynamic measures of the network (e.g. the Gibbs-Homology and cycle basis) as described in U.S. Provisional Application No. 62/165,879 to which this application claims priority. As described in U.S. Provisional Application No. 62/165,879 the user's inputs (transcriptomes, genomic alteration panels and/or whole genome sequencing) is processed for isolation of genes contributing to disease progression in order to focus the therapy design process on key pathways to analyze.

From this smaller set of genes, the system of one or more embodiments identifies the set of biological pathways where this smaller subset of genes is active. Of particularly interest are the genes that are located on biological pathways that are known to be relevant to cancer, or exhibit properties that may support cancer, as reflected in its knowledge base. The system of one or more embodiments then examines all protein-protein reactions which include the molecules of interest, those identified as important for the patient's specific molecular signature, and looks for bottlenecks and redundant reactions.

In one or more embodiments, a variety of plug-ins provide additional points of analysis, which are used by a meta-reasoner to score and weight the significance of each of the initially identified genes are being a likely successful target for metronomic intervention. The scoring is further updated by considering drug information, identifying which of the likely molecular targets has an FDA-approved drug for the disease indication, barring that, which drug has an FDA approval for other disease indications, and barring that, whether a potential experimental agent exists.

In one or more embodiments, this information is used to update the disease therapy model, and is further supplemented by considering contraindications by taking into account any secondary conditions or any overlapping toxicities of drug combinations.

In one or more embodiments, the system at this stage also considers whether the targeting of any of the molecular targets or the use of any drug(s) would lead to a phenotype that is incompatible with life. At each step, the system of one or more embodiments records and explicates its reasoning processes, allowing human users to be able to trace back the reasoning to source material or authoritative sources.

Based on this analysis, the system provides the user with a set of small protein-protein interaction networks, which include the target molecules and reactions on biological pathways that the model suggests would be most likely to be useful for therapy. The weightings for each such drug selection are presented, alongside the rationale for the decision. In one or more embodiments, using the system's Natural Language Generation capabilities, the system would provide an English (or any other language) explanation for each of the decisions in each of the options it presents.

In one or more embodiments, an expert panel such as a tumor board would then discuss the presented solutions, and in some cases update the model based on human input by including a novel contraindication, or incorporating information about novel drugs or gene-gene (molecular) interactions. In one or more embodiments, once the panel of experts validates a therapy for the patient, a user would indicate which set of molecular targets and drugs were used, and input the dosage, frequency and other details about the treatment plan.

In one or more embodiments, once the selected therapy is saved, the caregiver may decide to have the system generate a set of rationale needed for approval of the therapy by insurance companies. The caregiver would then administer the therapy to the patient according to the treatment plan, regularly documenting outcome measures to chart the patient's progress.

Each measure which includes, but is not limited to, disease imaging (2D, volumetric or other), severe adverse effects (SAE's) and additional biomarker evaluation, is input into the system, closing the iterative feedback loop and providing data updating the disease therapy model. In one or more embodiments, if the treatment plan is not progressing according to the caregiver's expectations, or if new information emerges, the caregiver may decide to refine or fail the therapy.

It would be apparent to one or ordinary skill in the art that in either case, the user action would update the disease model, capturing the rationale for why the treatment is not working as expected, and why an alternate approach is taken. In this manner, the system of one or more embodiments continues to grow and learn with each new designed therapy and treatment plan.

In one or more embodiments, much of the material that is taken as input described above exists scattered across multiple resources. The system provides the user with a consolidated view across these resources and domains, synthesizing information into clinically relevant view. A key challenge facing clinical oncologists is that they do not know how to interpret and make clinically actionable decisions based on genomic, proteomic and metabolomic information. The expertise required spans multiple disciplines and there is no one expert in all the fields. In one or more embodiments, the system acts as such an “expert”.

Compounding the problem of interpretation is that clinicians are overwhelmed by the amount of available and continuously evolving information. It would be apparent to one of ordinary skill in the art that it is humanly impossible to keep up with every possible journal article or database update. Furthermore, where such information exists, it is not readily accessible or has been created for consumption by other communities of interest.

For example, much of the genomic information is in databases or resources geared towards researchers involved in gene cloning, creation of transgenic animals etc., while proteomic resources are geared towards crystalografers, enzymatologists and other protein structure researchers. No resources exist to assist clinicians in keeping track of and make sense of the available relevant information. A number of resources are being developed for streamlining the enrollment of patients in clinical trials. However, there is no tool providing clinicians with guidance on developing safe, molecularly-targeted therapies based on the latest and well-curated information.

In one or more embodiments, an important component of the system is the feedback loop that establishes the link between patients treated by the therapy design strategy and the system's ability to aggregate and learn from these outcomes. Throughout the therapy design process, one or more embodiments of the invention is building an understanding of the following three domains:
1. Disease Biology Characterization;
2. Disease Pathway Analysis; and
3. Therapy Design.

Patient outcome data, as captured by measures such as disease bulk regression, toxicity and/or biomarker response, all allowing the system to refine its understanding and more accurately characterizing the three items above. In one or more embodiments, the outcome data that is of interest includes:

1. Disease imaging—is the disease burden responding?

a. 2D imaging of the disease

b. Volumetric imaging of the disease

c. Laboratory or other measures of the disease burden

2. Toxicity—how is the patients quality of life affected by the therapy
3. Biomarker evaluation—is the therapy performing in the anticipated manner
a. Measured across a number (of growing) molecular markers.

In one or more embodiments, the system takes all of these data points as inputs to chart the patient progress, and response to therapy. As the caregiver administers the therapy, the tool is able to continually update its own understanding of the disease and patient biology based on these inputs. Should the outcome data point to a conclusion that contradicts the assumptions in the patient/disease model, the tool would notify the caregiver and/or panel of experts of a misalignment of assumptions.

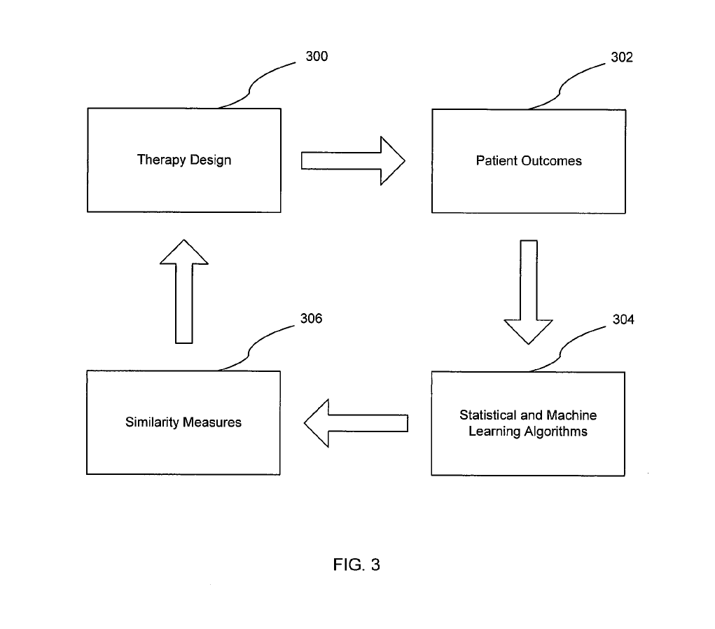

Alternatively, in one or more embodiments, the clinician and/or panel of experts may decide to refine or fail the therapy if the treatment plan is not progressing according to expectations or improving the patient's health. Each case that has been treated therefore informs future decisions about therapy, thereby tackling the “N of one” problem. In one or more embodiments, should the caregiver and/or panel of experts decide to refine the therapy, the important feedback loop mechanism mentioned above is utilized. An overview of the feedback loop mechanism of one or more embodiments is shown in FIG. 3.

FIG. 3 is a diagram in accordance to one or more embodiments. In one or more embodiments, as seen in FIG. 3, the feedback loop mechanism includes therapy design (300), patient outcomes (302), a statistical and machine learning algorithm (304), and an analysis and information of similarity measures (306).

In one or more embodiments, a therapy is designed (300) for a patient. Accordingly, the patient outcomes (302) including the therapy response measures are continuously collected, aggregated, and analyzed. The analyzed patient outcomes (302) are continuously applied to a statistical and machine learning algorithm (304) to derive similarity measures (306) between one patient's results with other patient's results.

Using one approach, the dataset created by each new patient is analyzed by proprietary statistical and machine learning techniques to identify patterns and reuse knowledge learned from one (or sets of) patient outcome(s) to new patients. The system in one or more embodiments employs a similarity measure that allows comparisons to be made across patients and disease models profiles. It would be apparent to one of ordinary skill in the art that it is possible to transfer knowledge learned from one patient outcome to another.

In one or more embodiments, as each new patient is inputted into the system, the process of developing a disease model and ultimately a treatment plan teaches the system a set of associations between specific genetic, proteomic and other patient and disease information, and the selected drugs and pathways.

In one or more embodiments, a method of deploying such a feedback mechanism involves extracting meta-genetic information to develop novel models about the connection of multiple molecules, pathways, and specific patient disease models. This meta-genetic information is employed to aid users in identifying similarities between new patients and those already within the system, allowing similar successful cases to be manually examined by the expert panels. In one or more embodiments, similarity can be measured by determining to what degree a patient's transcriptome and molecular alterations correspond to or overlap with identified meta-genes.

In one or more embodiments, another method of deploying a feedback mechanism involves casting each triple of patient model, disease model and treatment plan into an n-dimensional space, where characteristics of each of the preceding (such as the patient's transcriptome, proteome, pathway information, drug information, dosage information, etc.) are captured without interpretation as data points. In one or more embodiments, a semantic interpretation is deployed on each characteristic according to the underlying knowledge representation, where a set of reasoning engines attempt to construct a consistent model of all the patients, disease models, and treatment plans. Consequently, as each new patient-disease-model is input, the system checks to see whether the unique model produces any inconsistencies with previous treatments and disease models. If so, this will trigger a conditional belief revision process where the conflicting semantics of the models are highlighted for semi-automatic updating, in some cases resulting in an updating of the underlying knowledge representation.

Concurrently, in one or more embodiments of the invention, a variety of clustering algorithms are deployed on the un-interpreted patient-disease-treatment data to automatically extract multiple sets of features, enabling the clustering of patients, diseases, and treatments—both individually and combinatorial—into multiple groups and clusters. Patient outcome data is then used by both supervised and reinforcement learning algorithms to refine hybrid symbolic-statistical models, where each characteristic is initially connected to a semantic interpretation captured symbolically in the knowledge representation. This hybrid model includes layers of features extracted by the clustering algorithms overlaid on the semantic model, with numerically weighted associations between the symbols, the data points, and learned features. Then, in one or more embodiments, each new patient, based on the success or failure of a patient outcome given a disease model and treatment plan updates the numerical association of the data points within the n-dimensional space, either strengthening or weakening associations. This type of feedback mechanism, given enough model revision, may further trigger semi-automated belief revision of the knowledge representation component of the system. Consequently, at a high level, in such a way, the system is able to learn not just from successful or unsuccessful treatments, but any revision of the underlying disease model or treatment plan in accordance with one or more embodiments of the invention.

It would be apparent to one of ordinary skill the art that it is possible to transfer knowledge learned from one patient outcome to another. It would further be apparent to one of ordinary skill in the art that this feedback loop mechanism allows the information of the mutation and drugs, and the results of the therapies for multiple patients to be dynamically grouped. In one or more embodiments, this allows the system of one or more embodiments to continuously learn based on the information of each new patient entered resulting in the possibility that the system of one or more embodiments may notify the user if a previously attempted therapy has a higher or lower likelihood of success.

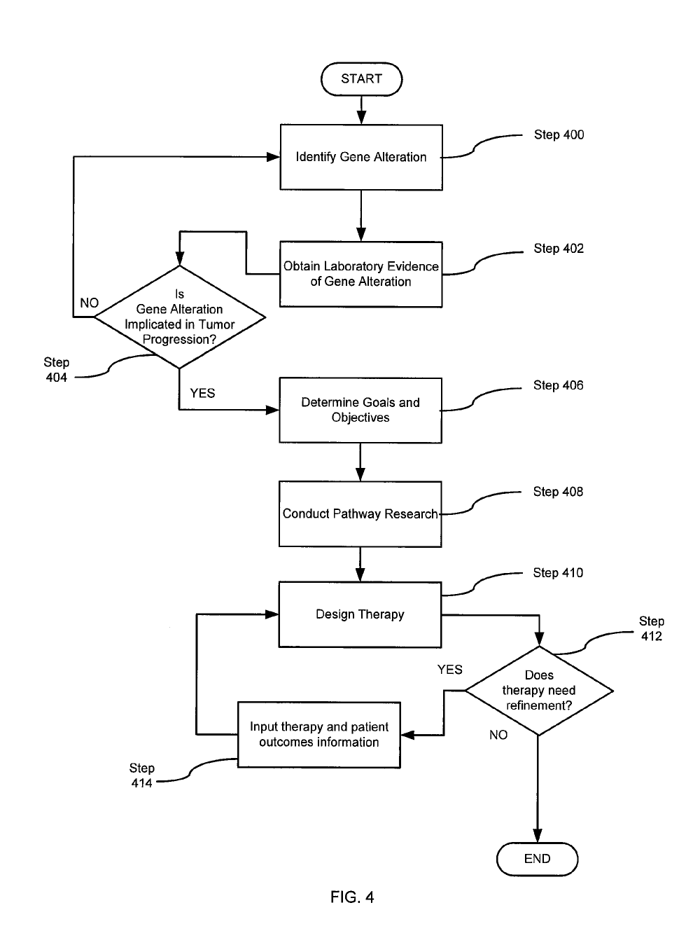

A detailed view of the feedback loop mechanism is shown in FIG. 4. Particularly, FIG. 4 shows a flow chart of a method in accordance to one or more embodiments. In one or more embodiments, the method in the flow charts in FIG. 4 involves identifying a gene alteration(s) (Step 400), obtaining laboratory evidence of the gene alteration(s) (402), determining if the gene alteration is implicated in tumor progression (Step 404), determining goals and objectives (Step 406), conducting pathway research (Step 408), designing a therapy (Step 410), and determining if the therapy needs refinement (412).

In one or more embodiments, in Step 404, if the gene alteration is not implicated in tumor progression, the method returns to Step 400 to identify a new gene alteration(s). In Step 412, if it is determined that the therapy does not need refinement, the feedback loop mechanism ends. However, if it is determined that the therapy does need refinement, the caregiver performs inputting of the information regarding the therapy and patient outcome (Step 414) and returns to Step 410 to design a new therapy based on the entered information.

In one or more embodiments, a simple example of this feedback loop mechanism is described below. For example, if a particular patient with a specific set of alterations is not responding to a combination therapy, the system of one or more embodiments would score that particular set of drugs/treatment plan lower for another patient with a similar set of mutations or target gene pathway networks. With each new patient, the system of one or more embodiments is constantly attempting to find new patterns and group patients into different similarity sets. With each iteration, the system of one or more embodiments refines the scores for how it evaluates new treatments based on its understanding of the biological characteristics of the disease, the pathway analysis, drug availability and overall therapy cost.

A number of machine learning technologies, trained on a model of disease, to index and interpret natural language sources (such as publications, journal articles, etc.) are deployed. Given that each of the scientific disciplines that inform our model are undergoing constant evolution—new gene functions, pathways and drugs are being discovered all the time—these algorithms are additionally deployed to constantly monitor novel research and incorporate new findings into our model.

In one or more embodiments, the operational model of the disease has embedded within it the metronomic, bio-marker driven, molecularly targeted combination therapy strategy. This operational model of the disease is streamlined to use the available information in the service of generating exactly such a therapy. To this end, a computer platform has been developed according to one or more embodiments, whereby a clinician can interact with our software (and the embedded model) to design a unique therapy based on the patient's unique molecular, genomic and proteomic information.

In one or more embodiments, the software system takes as input available information (ideally full transcriptome and proteome, though in the worst case, it can work with candidate or panel-based genomic tests), and walks a clinician through the therapy design process, as described above. It should be noted, that the computational model of the disease allows us to, at each step, explain exactly the scientific rationale and grounding for every decision made within the system. Ultimately, this means that the Natural Language Generation capabilities may automatically author the rationale to support a specific therapy for a patient.

One or more embodiments include several items:
1. Strategy for metronomic, bio-marker driven, molecularly targeted combination therapy;
2. Computational, operational model of disease;
3. Machine learning algorithms to supplement and constantly feed the model with updates; and/or
4. Software system that combines 1-3 above and allows clinicians and expert panels to interactively design unique patient therapies.

It would be apparent to one of ordinary skill in the art that the software of one or more embodiments above is well suited to integrate with insurance companies to automatically assess the rationale provided by clinicians for given therapies in accordance with one or more embodiments of the invention. In one or more embodiments, the software is also well suited to be used as a teaching tool in universities, medical centers and continuing education to popularize integration of genomic, proteomic, metabolomic and pharmacogenomic information use in clinical decision making and to enhance the provision and/or delivery of next generation sequencing services.

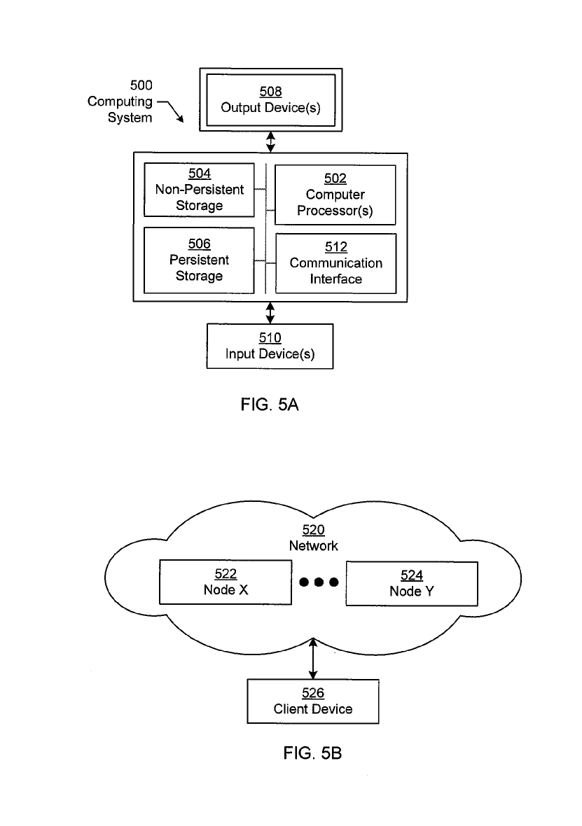

FIGS. 5A and 5B show a computing system in accordance with one or more embodiments of the technology.

Embodiments of the invention may be implemented on a computing system. Any combination of mobile, desktop, server, router, switch, embedded device, or other types of hardware may be used. For example, as shown in FIG. 5A, the computing system (500) may include one or more computer processors (502), non-persistent storage (504) (e.g., volatile memory, such as random access memory (RAM), cache memory), persistent storage (506) (e.g., a hard disk, an optical drive such as a compact disk (CD) drive or digital versatile disk (DVD) drive, a flash memory, etc.), a communication interface (512) (e.g., Bluetooth interface, infrared interface, network interface, optical interface, etc.), and numerous other elements and functionalities.

The computer processor(s) (502) may be an integrated circuit for processing instructions. For example, the computer processor(s) may be one or more cores or micro-cores of a processor. The computing system (500) may also include one or more input devices (510), such as a touchscreen, keyboard, mouse, microphone, touchpad, electronic pen, or any other type of input device.

The communication interface (512) may include an integrated circuit for connecting the computing system (500) to a network (not shown) (e.g., a local area network (LAN), a wide area network (WAN) such as the Internet, mobile network, or any other type of network) and/or to another device, such as another computing device.

Further, the computing system (500) may include one or more output devices (508), such as a screen (e.g., a liquid crystal display (LCD), a plasma display, touchscreen, cathode ray tube (CRT) monitor, projector, or other display device), a printer, external storage, or any other output device. One or more of the output devices may be the same or different from the input device(s). The input and output device(s) may be locally or remotely connected to the computer processor(s) (502), non-persistent storage (504), and persistent storage (506). Many different types of computing systems exist, and the aforementioned input and output device(s) may take other forms.

Software instructions in the form of computer readable program code to perform embodiments of the invention may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (500) in FIG. 5A may be connected to or be a part of a network. For example, as shown in FIG. 5B, the network (520) may include multiple nodes (e.g., node X (522), node Y (524)). Each node may correspond to a computing system, such as the computing system shown in FIG. 5A, or a group of nodes combined may correspond to the computing system shown in FIG. 5A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (500) may be located at a remote location and connected to the other elements over a network.

Although not shown in FIG. 5B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (522), node Y (524)) in the network (520) may be configured to provide services for a client device (526). For example, the nodes may be part of a cloud computing system. The nodes may include functionality to receive requests from the client device (526) and transmit responses to the client device (526). The client device (526) may be a computing system, such as the computing system shown in FIG. 5A. Further, the client device (526) may include and/or perform all or a portion of one or more embodiments of the invention.

The computing system or group of computing systems described in FIGS. 5A and 5B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process.

Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a GUI on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system in FIG. 5A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail—such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system of FIG. 5A, while performing one or more embodiments of the invention, may perform data comparison. Data comparison may be used to compare two or more data values (e.g., A, B). For example, one or more embodiments may determine whether A>B, A=B, A !=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system in FIG. 5A may implement and/or be connected to a data repository. For example, one type of data repository is a database. A database is a collection of information configured for ease of data retrieval, modification, re-organization, and deletion. Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g. join, full join, count, average, etc.), sort (e.g. ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system of FIG. 5A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions present only a few examples of functions performed by the computing system of FIG. 5A and the nodes and/or client device in FIG. 5B. Other functions may be performed using one or more embodiments of the invention.

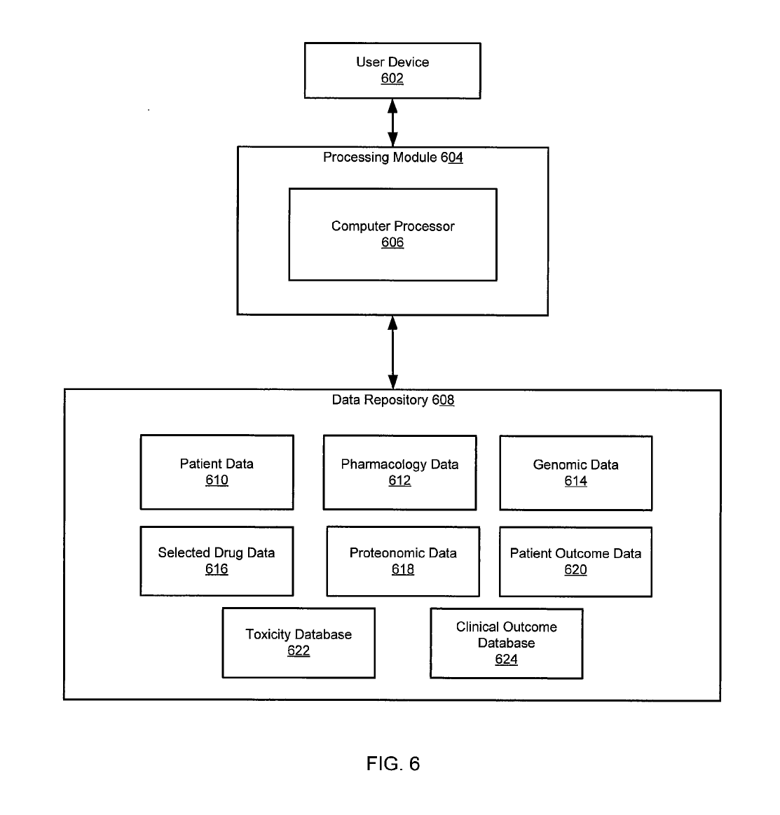

FIG. 6 shows a schematic diagram of a system in accordance with one or more embodiments. The system for selecting a protein target for therapeutic application includes (i) a processing module (604) including a computer processor (606) configured to execute instructions configured to: access information associated with a patient and a reference biological network database; generate, using the information associated with the patient and the reference biological network database, a disease model; identify, from the disease model, a molecular target; identify, from the molecular target, a drug for the patient; generate, based on the drug for the patient, a treatment plan for the patient; and repetitively generate, based on repetitively inputting a patient outcome from the treatment plan into a feedback loop mechanism, a different treatment plan for the patient based on either the molecular target or a different molecular target. and (ii) a user device (602) configured to present the protein target to a user. The system may further include a data repository (608) configured to store the patient data (610), the pharmacology data (612), the genomic data (614), the selected drug data (616), the proteonomic data (618), the patient outcome data (620), the toxicity database (622), and the clinical outcome database (624).

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method for therapeutic application, comprising:
accessing information associated with a patient and a reference biological network database;
generating, using the information associated with the patient and the reference biological network database, a disease model;
identifying, from the disease model, a molecular target;
identifying, from the molecular target, a drug for the patient;
generating, based on the drug for the patient, a treatment plan for the patient; and
repetitively generating, based on repetitively inputting a patient outcome from the treatment plan into a feedback loop mechanism, a different treatment plan for the patient based on either the molecular target or a different molecular target.

2. The method of claim 1, further comprising:
displaying the molecular target to a user.

3. The method of claim 1, further comprising:
repetitively storing, in a data repository, the information associated with the patient, the reference biological network database, the disease model, the molecular target data, and a data for the drug for the patient.

4. The method of claim 3, wherein the information associated with a patient and the reference biological network database is at least one from a group consisting of genomic, proteomic, transcriptomic, histological, metabolomic, and epigenetic network pathway data.

5. The method of claim 3, wherein the information associated with a patient and the reference biological network database is one from a group consisting: an academic database, a public database, and a private database.

6. The method of claim 3, wherein the information associated with the patient is processed using a computational and mathematical analysis from a group consisting of Gibbs-Homology, cycle-basis analysis, and prioritization of relevant gene networks.

7. The method of claim 3, wherein the disease model is generated by mapping at least one from the group consisting of genomic, proteomic, transcriptomic, histological, metabolomic, and epigenetic information to at least one from the group consisting of genomic, proteomic, transcriptomic, histological, metabolomic, and epigenetic network pathway data;

8. The method of claim 3, wherein the drug for the patient is selected based on the combination of a drug evaluation process, a molecular target and drug filter process, a host biology and tumor model process, and a tumor board evaluation and refinement process.

9. The method of claim 3, wherein the treatment plan comprises a drug dosage and a frequency and the different treatment plan comprises a different drug dosage and a different frequency.

10. The method of claim 3, wherein the results are based on a combination of therapy administration and patient outcome data.

11. The method of claim 3, wherein the feedback loop mechanism continuously collects, aggregates, and analyzes the treatment plan and the patient outcome using a statistical and machine learning algorithm to derive similarity measures between patients, mutations, and drugs.

12. A computing system for therapeutic application, comprising:
a processing module comprising a computer processor with circuitry configured to execute instructions configured to:
access information associated with a patient and a reference biological network database;
generate, using the information associated with the patient and the reference biological network database, a disease model;
identify, from the disease model, a molecular target;
identify, from the molecular target, a drug for the patient;
generate, based on the drug for the patient, a treatment plan for the patient; and
repetitively generate, based on repetitively inputting a patient outcome from the treatment plan into a feedback loop mechanism, a different treatment plan for the patient based on either the molecular target or a different molecular target.

13. The system of claim 12, further comprising:
a data repository configured to repetitively store the information associated with the patient, the reference biological network database, the disease model, the molecular target data, and a data for the drug for the patient.

14. A non-transitory computer-readable medium having instructions stored thereon that, in response to execution by the computer system, cause the computer system to perform operations comprising:
accessing information associated with a patient and a reference biological network database;
generating, using the information associated with the patient and the reference biological network database, a disease model;
identifying, from the disease model, a molecular target;
identifying, from the molecular target, a drug for the patient; and
generating, based on the drug for the patient, a treatment plan for the patient
repetitively generating, based on repetitively inputting a patient outcome from the treatment plan into a feedback loop mechanism, a different treatment plan for the patient based on either the molecular target or a different molecular target.

15. The non-transitory computer-readable medium of claim 14, further comprising:
a data repository configured to repetitively store repetitively storing, in a data repository, the information associated with the patient, the reference biological network database, the disease model, the molecular target data, and a data for the drug for the patient.
